(19) United States
(12) Patent Application Publication
(10) Pub. No.: US 2013/0236949 A1
(43) Pub. Date: Sep. 12, 2013

(54) COMPOSITION AND METHOD FOR BIOREMEDIATION OF DEICING FLUIDS

(71) Applicant: METHOD4 LLC, King of Prussia, PA (US)

(72) Inventor: Raj J. Mehta, Wayne, PA (US)

(73) Assignee: Method4 LLC, King of Prussia, PA (US)

(21) Appl. No.: 13/873,499

(22) Filed: Apr. 30, 2013

Related U.S. Application Data
(63) Continuation of application No. 13/081,824, filed on Apr. 7, 2011, now abandoned.
(60) Provisional application No. 61/321,695, filed on Apr. 7, 2010.

(51) Int. Cl. A62D 3/02; C12N 1/20

(52) U.S. Cl. CPC A62D 3/02; C12N 1/20
USPC 435/252.4; 435/262.5

(57) ABSTRACT

The present invention is a kit and method for bioremediation of deicing fluids containing glycol. Using a combination Bacillus, Pseudomonas, yeast and filamentous fungi strains selected for the ability to grow on high levels of glycol, bioremediation of airport runoff is provided.

This application is a continuation of U.S. patent application Ser. No. 13/081,824 filed Apr. 7, 2011, which claims priority of U.S. Provisional Patent Application No. 61/321,695 filed Apr. 7, 2010, the teachings of which are incorporated herein by reference in their entireties.

### BACKGROUND OF THE INVENTION

Glycol-based aircraft deicing fluids are routinely used at many airports to control ice formation on aircraft before takeoff. There are two general types of deicing fluids. Type I which is composed of glycol (propylene glycol, ethylene glycol or diethylene glycol), water, coloring agents, and corrosion inhibitor (nitrates, silicates, etc.) and is used to physically remove snow or ice adhering to aircraft surfaces. Type II is essentially Type I that contains synthetic polymers, allowing it to adhere to aircraft surfaces for extended lengths of time. During deicing operations, a significant portion of excess deicing fluid runs off the aircraft where it may enter storm drains and nearby surface water and cause environmental damage. Aircraft deicing fluid exhibits biochemical oxygen demands that often exceed that of domestic wastewater and contains chemical additives (e.g., surfactants and corrosion inhibitors) that may be toxic to aquatic life at high concentrations. As a result, the control of deicing runoff is now being mandated by both federal and state regulatory agencies to protect both human health and the environment. With these regulations, many airports face dual challenges of simultaneously maintaining public safety and protecting the environment.

While biodegradation of propylene glycol-based deicing fluids has been suggested (Bausmith & Neufeld (1999) Water. Environ. Res. 71, 459-464), with anaerobic and aerobic biodegradation at low levels of glycol (2-3%) by a few bacterial strains, spent deicing fluid contains much higher levels of glycols plus inhibitors. Because glycols are also used as preservatives, they typically inhibit microbial growth. Therefore, there is a need for improved methods for bioremediation of deicing fluids.

### SUMMARY OF THE INVENTION

The present invention features a kit and method for the bioremediation of deicing fluids. The present invention is based upon the use of at least one isolated Bacillus species, at least one isolated Pseudomonas species, at least one isolated yeast and at least one isolated filamentous fungus, each of which is selected for an enhanced ability to degrade a glycol. In some embodiments, the strains are selected for enhanced growth on medium containing at least 15% glycol and up to 30% glycol. In other embodiments, the strains are selected for enhanced growth on medium containing 15% deicing fluid containing inhibitors. In still other embodiments, the kit and method include the addition of one or more of amino acids, B complex, or minerals from soluble seaweed. Still further, other embodiments include the selection of strains for an enhanced ability to degrade a glycol at 5° C. or 30° C.-45° C.

### DETAILED DESCRIPTION OF THE INVENTION

Each year large quantities of propylene glycol (PG) and ethylene glycol (EG)-based aircraft deicing fluids (ADF) are used to de-ice aircraft. On the average, it takes 500 to 1000 gallons of ADF to deice a large commercial aircraft and a medium-sized airport may use over 264,000 gallons of fluid over the entire winter season. Given the environmental concerns associated with wintertime aircraft operations, the present invention features a solution for the management of deicing chemical runoff by providing a combination of microorganisms selected for their ability to bioremediate high concentrations of glycols.

The present invention features a combination of microorganisms selected for their ability to rapidly grow on high concentrations of deicing fluid. Microorganisms of particular use include those which can be manipulated under laboratory conditions, yet readily grow under environmental conditions. Specifically, the present invention embraces a combination of at least one isolated Bacillus, at least one isolated Pseudomonas, at least one isolated yeast and at least one isolated filamentous fungus.

Facultative bacteria belonging to the Bacillus and Pseudomonas species are desirable because they consume organic waste thousands of times faster than the types of bacteria that are naturally present, they grow and reproduce easily, are non-pathogenic, and do not produce foul odors or gas. These bacteria can be cultured on a liquid or dry agar and can be freeze-dried, leaving them in a state of suspension. They remain alive and will function normally as soon as they are rehydrated and put into an acceptable environment. Examples of Bacillus species include, but are not limited to, B. licheniformis, B. subtilis, B. cereus, and B. circulans. Examples of Pseudomonas species include, but are not limited to, P. aeruginosa, P. putida, P. fluorescens and P. stutzeri.

Like bacteria, yeast can be readily manipulated under laboratory settings and are of use in the present invention. Examples of yeast include, but are not limited, Saccharomyces cerevisiae, Schizosaccharomyces pombe, Pichia (e.g., P. guilliermondii, P. ciferrii, P. pastoris or P. sydowiorum), Candida catenulate, and C. albicans.

Filamentous fungi are also embraced by this invention. These include fungi from the genera Aspergillus, Neurospora, Phanerochaete (e.g., P. chrysosporium), Allescheriella, Stachybotrys, and Phlebia.

Strains of use herein can be isolated from the environment or be conventional laboratory strains. Desirably, the strains are isolated and purified so as to be homogenous to a particular bacterium or fungus.

In so far as the concentration of glycols can be high in areas of need of remediation, particular embodiments of this invention feature bacteria and fungi that have been selected for an enhanced ability to grow on medium containing high concentrations of glycol. In this respect, bacteria and fungi with a 10%, 20%, 30%, 40, 50%, or more increase in the rate of growth on medium containing high levels of glycol (e.g., at least 15%) glycol, as compared to strains that have not been selected for enhanced growth, are of use in this invention. Desirably, the selection medium has at least 15% glycol, with particular embodiments embracing glycol present in the range of 15-30%. The glycol can be added to medium in purified form, e.g., isolated and purified propylene glycol, ethylene glycol or diethylene glycol, or alternatively be provided in the form of deicing fluid containing inhibitors.

In so far as it may be desirable to bioremediate soil during the winter or summer, particular embodiments of this invention further include the selection of bacteria and fungi that can bioremediate or degrade glycol under temperature extremes including, but not limited to, temperatures as low as 5° C. as well as temperatures in the range of 30° C. to 45° C.

To facilitate growth of the microorganisms of this invention under environmental conditions, some embodiments of this invention include providing with the microorganism one or more of amino acids, B complex, and/or minerals isolated or obtained from seaweed.

Because airport runoff can contain other compounds such as urea, calcium magnesium acetate, potassium acetate, potassium formate, sodium acetate, and sodium formate, some embodiments of this invention provide for the growth in, or degradation of, these compounds as well by the microorganisms of this invention.

What is claimed is:

1. A kit for bioremediation of deicing fluid comprising at least one isolated Bacillus species, at least one isolated Pseudomonas species, at least one isolated yeast and at least one isolated filamentous fungus, each of which is selected for an enhanced ability to degrade a glycol.

2. The kit of claim 1, wherein the strains are selected for enhanced growth on medium comprising at least 15% glycol.

3. The kit of claim 2, wherein the glycol is present in the growth medium at a concentration ranging from 15% to 30%.

4. The kit of claim 1, wherein the strains are selected for enhanced growth on medium comprising 15% deicing fluid containing inhibitors.

5. The kit of claim 1, further comprising one or more of amino acids, B complex, or minerals from soluble seaweed.

6. The kit of claim 1, wherein the strains are further selected for an enhanced ability to degrade a glycol at 5° C.

7. The kit of claim 1, wherein the strains are further selected for an enhanced ability to degrade a glycol at 30° C. to 45° C.

8. A method for bioremediation of deicing fluid-contaminated sample comprising contacting a deicing fluid-contaminated sample with a combination of at least one isolated Bacillus species, at least one isolated Pseudomonas species, at least one isolated yeast and at least one isolated filamentous fungus, each of which is selected for an enhanced ability to degrade a glycol, so that the deicing fluid-contaminated sample is bioremediated.

9. The method of claim 8, wherein the method further comprising contacting the sample with one or more of amino acids, B complex, or minerals from soluble seaweed.
